(19) United States
(12) Patent Application Publication
(10) Pub. No.: US 2025/0138309 A1
(43) Pub. Date: May 1, 2025

(54) DEVICES FOR APPLYING ELECTROMAGNETIC ENERGY WITH PRISMATIC BEAM STEERING

(71) Applicant: SOLTA MEDICAL IRELAND LIMITED, DUBLIN (IE)

(72) Inventor: Jessica WEST, Bothell, WA (US)

(21) Appl. No.: 18/864,150

(22) PCT Filed: May 10, 2023
(86) PCT No.: PCT/EP2023/062511

§ 371 Date: Nov. 8, 2024

Related U.S. Application Data
(60) Provisional application No. 63/340,100, filed on May 10, 2022.

(51) Int. Cl. G02B 27/00; A61B 18/20

(52) U.S. Cl. CPC G02B 27/0068; A61B 2018/202; A61B 18/203

(57) ABSTRACT

Some embodiments relate to Devices-a device for applying electromagnetic energy and related methods. A device includes a source configured to provide a beam of electromagnetic energy, a collimator configured to receive the beam from the source, a first optical element, and a second optical element having an optical access. The first optical element is positioned between the collimator and the second optical element. The first optical element is configured to correct for misalignment of the beam relative to the optical axis of the second optical element.

### CROSS-REFERENCE TO RELATED APPLICATIONS

This application is a national phase filing under 35 C.F.R. § 371 of and claims priority to PCT Patent Application No. PCT/EP2023/062511, filed on May 10, 2023, which claims the priority benefit under 35 U.S.C. § 119 of U.S. Provisional Patent Application No. 63/340,100, filed on May 10, 2022, the contents of which are hereby incorporated in their entireties by reference.

The disclosure generally relates to the treatment of tissue with electromagnetic energy and, more particularly, to devices for applying electromagnetic energy and related methods.

Electromagnetic energy, particularly laser energy, may be used as an adaptable tool in medicine to achieve desired outcomes in treated tissue. For example, lasers and other forms of electromagnetic energy may be used to treat common dermatological conditions. Forms of electromagnetic energy may also be used to improve appearance by resurfacing the skin, by remodeling the different layers of skin to eliminate wrinkles, and/or by tightening the skin.

Generally, skin resurfacing is a process by which the top layers of the skin are damaged in order to promote the development of new, more youthful-looking skin and to stimulate the generation and growth of new skin. For example, a laser may be used to create treatment zones containing damaged skin that, upon healing, operates to rejuvenate the skin tissue. Generally, lasers that are used for skin resurfacing operate at a wavelength that is absorbed by one of the natural chromophores in the skin, such as water. If water is the primary chromophore, cellular and interstitial water absorbs light energy and transforms the light energy into thermal energy.

Improved devices for applying electromagnetic energy and related methods are needed.

### SUMMARY

In an embodiment of the presently disclosed subject matter, a device comprises a source configured to provide a beam of electromagnetic energy, a collimator configured to receive the beam from the source, a first optical element having an optical axis, and a second optical element. The first optical element is positioned between the collimator and the second optical element. The first optical element is configured to correct for misalignment of the beam relative to the optical axis of the second optical element.

In an embodiment of the presently disclosed subject matter, a method comprises directing a beam of electromagnetic energy though a collimator to a first optical element, and rotating a first prism of the first optical element relative to a second prism of the first optical element to correct for misalignment of the beam relative to an optical axis of a second optical element downstream from the first optical element.

### BRIEF DESCRIPTION OF THE DRAWINGS

The accompanying drawings, which are incorporated in and constitute a part of this specification, illustrate various embodiments of the presently disclosed subject matter and, together with
a general description of the presently disclosed subject matter given above and the detailed description of the embodiments given below, serve to explain the embodiments of the presently disclosed subject matter. In the drawings, like reference numerals refer to like features in the various views.

FIG. 1 is a diagrammatic view of a device configured to apply electromagnetic energy in accordance with embodiments of the presently disclosed subject matter.

FIG. 2 is an exploded view of the collimator and the prisms of FIG. 1.

FIG. 3 is an assembled view of the collimator and the prisms of FIG. 1.

FIG. 4 is a diagrammatic view illustrating the principle of operation of the prisms.

### DETAILED DESCRIPTION

Unless defined otherwise, all technical and scientific terms used herein have the same meaning as is commonly understood by a person having ordinary skill in the art to which this presently disclosed subject matter pertains. All patents and publications referred to herein are incorporated by reference in their entireties.

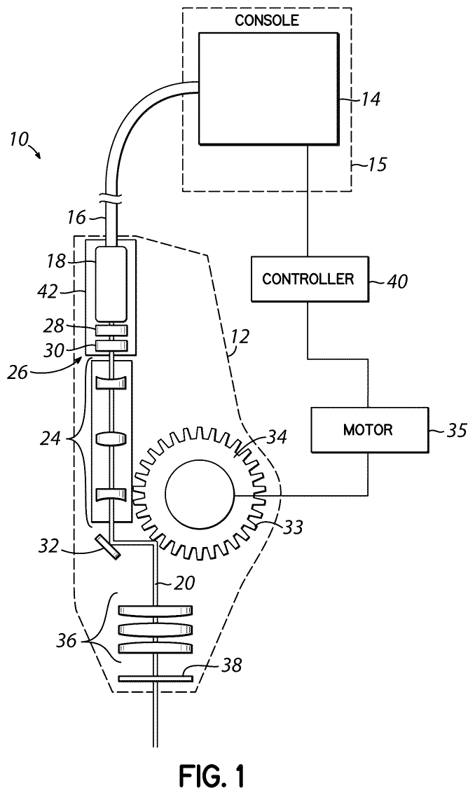

With reference to FIGS. 1-3, and in accordance with embodiments of the presently disclosed subject matter, a device 10 is configured to apply electromagnetic energy, such as laser energy, to tissue, such as skin tissue. For example, the electromagnetic energy applied by the device 10 may be laser energy that is used to perform a dermatological treatment of a targeted portion of skin tissue and, more specifically, to perform an ablative or non-ablative fractional skin resurfacing treatment of only a fraction of the skin tissue in the targeted portion. A fractional skin resurfacing treatment may produce thousands of microscopic treatment zones containing damaged skin cells in the targeted portion, as well as untreated areas containing undamaged that surround the treatment zones and serve to provide a rapid healing response and o produce new tissue.

The device 10 may include a handpiece 12. The handpiece 12 includes a housing and may be configured and dimensioned to be manually grasped and manipulated relative to a surface, such as a surface of a targeted portion of skin tissue. An electromagnetic energy source 14, which may be located at a console 15 that is remote from the handpiece 12, may be connected to the handpiece 12 by an optical fiber 16. The electromagnetic energy source 14 may include one or more lasers each configured to output a beam 20 containing electromagnetic radiation (i.e., coherent light) at a given wavelength. For example, the electromagnetic energy source 14 may include an erbium laser, a carbon dioxide laser, and/or a thulium laser. As a specific example, the electromagnetic energy source 14 may include an erbium laser configured to output light at a wavelength of about 1550 nanometers and a thulium laser configured to output light at a wavelength of about 1927 nanometers. In an embodiment, the electromagnetic energy source 14 may be a fiber-coupled laser diode that couples the light generated by the laser diode directly into the optical fiber 16.

The electromagnetic energy source 14 generates a beam 20 of electromagnetic energy that is transmitted via the optical fiber 16 from the electromagnetic energy source 14 to the handpiece 12. A collimator 18 may be coupled to an end of the optical fiber 16 and may be located inside the handpiece 12. The collimator 18 may be configured to collimate the beam 20 of electromagnetic energy received from the electromagnetic energy source 14. In that regard, the collimator 18 may reduce the beam divergence by causing the rays of the beam 20 to become more aligned in a specific direction (e.g., more parallel rays) and/or by causing the spatial cross-section of the beam 20 to become smaller. In an embodiment, the collimator 18 may be a component of a fiber-coupled laser diode and may terminate the optical fiber 16.

The beam 20 is launched from the collimator 18 with a reduced beam divergence into free space inside the handpiece 12 and directed in a light path toward an optical element 26 (i.e., a first optical element 26). In the representative embodiment, the optical element 26 may include a prism 28 and a prism 30 that are positioned adjacent to each other. The beam 20 exiting the optical element 26 is directed toward an optical element 24 (i.e., a second optical element 24) comprised of lenses and positioned downstream in the light path from the optical element 26.

The beam 20 exiting the second optical element 24 is reflected by a mirror 32 toward a scanner wheel 34, also referred to as a Broome wheel. The scanner wheel 34, which has an axis of rotation and is powered by a motor 35 to spin about the axis of rotation, has reflective elements 33 arranged about its circumference. The reflective elements 33 are configured to deflect the beam 20 to a set of lens elements 36, which focus the beam 20 through an output window 38 and toward a tissue surface to be treated by the electromagnetic energy. The operation of the scanner wheel 34 generates beamlets from the beam 20 and outputs the beamlets to the lens elements 36 in a pattern, which may be a fractionated pattern represented by a plurality of “dots” at the tissue surface resulting from the beamlets contacting the tissue surface. In some embodiments, the beamlets contained in the beam 20 are projected from the device 10 with the pattern, which may be used to generate the microscopic treatment zones in only a fraction of the targeted skin tissue.

The handpiece 12 may be gripped by a technician and moved relative to a tissue surface, such as a skin surface, to provide the dermatological treatment. A treatment tip may be applied to the device 10 during the dermatological treatment and may include, for example, a set of rollers that facilitates movement relative to the tissue surface receiving the electromagnetic energy.

A controller 40 can be accessed by the user through a user interface to control operation of the motor 35 rotating the scanner wheel 34 and to select appropriate operational parameters for the electromagnetic energy source 14. For example, the controller 40 can be used to control operational parameters of the electromagnetic energy source 14, such as the wavelength, pulse energy, pulse shape, pulse repetition rate, and pulse duration of the beam 20 transmitted by the optical fiber 16 to the handpiece 12.

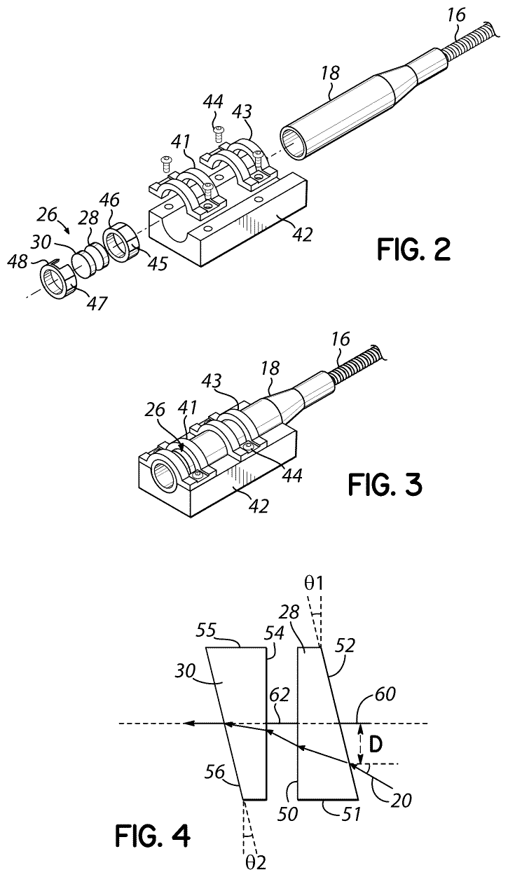

As best shown in FIG. 2, the prism 28 may be positioned and held inside a sleeve 46, and the prism 30 may be positioned and held inside a sleeve 48. In an embodiment, the prism 28 may be held irrotationally inside the sleeve 46, and the prism 30 may be held irrotationally inside the sleeve 48. For example, the prisms 28, 30 may be affixed by adhesive bonds to the sleeves 46, 48. In an alternative embodiment, the prisms 28, 30 may be affixed without adhesive in the sleeves 46, 48. In an embodiment, the prisms 28, 30 may be comprised of an optically-transparent material, such as fused silica, and the sleeves 46, 48 may be comprised of a metal, such as brass. The sleeves 46, 48 are open-ended so that the optical axes and light paths in a cone about the optical axes of the prisms 28, 30 are not obstructed.

The sleeves 46, 48 holding the prisms 28, 30 and the collimator 18 may be positioned and held inside of a set of clamps 41, 42, 43. The clamps 41, 42, 43 have a clamped state that can be established by tightening a set of fasteners 44 and an unclamped state that can be established by loosening the fasteners 44. The positions of the prisms 28, 30 and the collimator 18 relative to the clamps 41, 42, 43 and relative to each other are fixed in the clamped state. The sleeves 46, 48 can be rotated relative to each other when the clamps 41, 42, 43 are in the unclamped state, which permits the relative rotational orientation of the prisms 28, 30 to be changed.

The sleeves 46, 48 are positioned adjacent to each other inside of the clamps 41, 42, 43 such that the prisms 28, 30 are also positioned adjacent to each other. The sleeves 46, 48 may be rotated relative to each other in order to change the relative orientation of the prisms 28, 30, and then the rotational orientations inside the clamps 41, 42, 43 may be fixed by tightening the fasteners 44 to clamp the clamps 41, 42, 43. Slots 45 are arranged about a circumference of the sleeve 46, and slots 47 are arranged about a circumference of the sleeve 48. The sleeve 46 may be rotated inside the unclamped clamps 41, 42, 43 by engaging a tip of a tool with one of the accessible slots 45, and the sleeve 48 may be independently rotated inside the unclamped clamps 41, 42, 43 by engaging a tip of a tool with one of the accessible slots 47.

The optical element 26 is configured to provide prismatic beam steering that compensates for misalignment of the beam 20 exiting the collimator 18 relative to an optical axis of one or more of the lens within second optical element 24. In an embodiment, the prisms 28, 30 may be cylindrical prisms, such as Risley prisms, that can be used to steer the beam 20 by redirecting rays of light by refraction. Rotating the prisms 28, 30 relative to each other provides steering in order to correct any angular misalignment of the beam 20 between the collimator 18 and the second optical element 24, which is downstream from the optical element 26. For example, the beam 20 may be steered by the prisms 28, 30 to be aligned with the optical axis of the second optical element 24 and one or more of the lenses therein. The prisms 28, 30 are fixed in position at a correct angular alignment after correction of any angular misalignment.

As referred to herein, a “correct angular alignment” is a resulting angular alignment of the prisms 28, 30 such that the beam 20, steered by prisms 28, 30, substantially coincides with the optical axis of the second optical element 24. By contrast, if the prisms 28, 30 are not in the correct angular alignment, then the beam 20 will not substantially coincide with the optical axis of the second optical element 24 resulting in angular misalignment. In an embodiment, the beam 20 may be centered or substantially centered on the optical axis of the second optical element 24.

A recurrence of an angular misalignment, for example during use of the device 10, can be corrected by loosening the fasteners 44 to place the clamps 41, 42, 43 in their unclamped state so that the sleeves 46, 48 are freed to rotate and then performing an updated alignment routine involving relative rotation of the prisms 28, 30.

With reference to FIG. 4, the prism 28 has a surface 50, a surface 52 opposite to the surface 50, and a surface 51 connecting the surfaces 50, 52. The surfaces 50, 52 may be the disk-shaped bases of a cylinder and have a round cross-section, the surface 51 may be cylindrical, and the surfaces 50, 52 may be polished and planar. The prism 30 has a surface 54, a surface 56 opposite to the surface 54, and a surface 55 connecting the surfaces 54, 56. The surfaces 54, 56 may be the disk-shaped bases of a cylinder and have a round cross-section, the surface 55 may be cylindrical, and the surfaces 54, 56 may be polished and planar. The surface 50 of the prism 28 may be positioned adjacent to the surface 54 of the prism 30, and the surface 52 of the prism 28 may be positioned adjacent to the collimator 18 (FIG. 1). The surface 50 of the prism 28 and the surface 54 of the prism 30 may be located in parallel planes. In some embodiments, the surfaces 50 and 54 may be positioned in contact with each other.

The surface 51 of the prism 28 may be adhesively bonded to a cylindrical interior surface of the sleeve 46 (FIG. 2), and the surface 55 of the prism 30 may be adhesively bonded to a cylindrical interior surface of the sleeve 48 (FIG. 2). An anti-reflection coating may be applied to the surfaces 50, 52 of the prism 28 and to the surfaces 54, 56 of the prism 30.

The surface 52 of the prism 28 is inclined at a shallow wedge angle θ1, which is exaggerated for purposes of illustration in FIG. 4, relative to the surface 50 of the prism 28. The surface 56 of the prism 30 is inclined at a shallow wedge angle θ2, which is also exaggerated for purposes of illustration in FIG. 4, relative to the surface 54 of the prism 30. In an embodiment, the wedge angles of the surfaces 52, 56 may be greater than 0 degrees and less than or equal to about 3 degrees. In an embodiment, the wedge angles of the surfaces 52, 56 may be equal to about 0.5 degrees. In an embodiment, the wedge angle of the surface 52 may be equal to the wedge angle of the surface 56.

The prism 28 has an axis of rotation 60 and the prism 30 also has an axis of rotation 62 that may be aligned parallel to the axis of rotation 60. In an embodiment, the axis of rotation 60 may coincide with the optical axis of the prism 28, and the axis of rotation 62 may coincide with the optical axis of the prism 30. Rotating one or both of the prisms 28, 30 will change the direction of the beam 20. For example, the beam 20 may be laterally displaced by a distance D from the axis of rotation 60 and therefore misaligned when arriving from the collimator 18 at the surface 52 of the prism 28. The prism 28 deflects the misaligned beam 20 laterally in a direction toward the axis of rotation 60, and the beam 20 exiting the surface 50 of the prism 28 then encounters the surface 54 of the prism 30. When the prisms 28, 30 are rotationally aligned to compensate for the offset D, the prism 30 redirects the beam 20 to exit the surface 56 in a direction parallel or substantially parallel to the axis of rotation 62, which may be aligned with the optical axis of the second optical element 24 and one or more lenses therein (FIG. 1).

A feature “connected” or “coupled” to or with another feature may be directly connected or coupled to or with the other feature or, instead, one or more intervening features may be present. A feature may be “directly connected” or “directly coupled” to or with another feature if intervening features are absent. A feature may be “indirectly connected” or “indirectly coupled” to or with another feature if at least one intervening feature is present. A feature “on” or “contacting” another feature may be directly on or in direct contact with the other feature or, instead, one or more intervening features may be present. A feature may be “directly on” or in “direct contact” with another feature if intervening features are absent. A feature may be “indirectly on” or in “indirect contact” with another feature if at least one intervening feature is present. Different features may “overlap” if a feature extends over, and covers a part of, another feature with either direct contact or indirect contact.

References herein to terms modified by language of approximation, such as “about”, “approximately”, and “substantially”, are not to be limited to the precise value specified. The language of approximation may correspond to the precision of an instrument used to measure the value and, unless otherwise dependent on the precision of the instrument, may indicate a range of +/−10% of the stated value(s).

The transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed presently disclosed subject matter. All embodiments described herein that encompass the presently disclosed subject matter can, in alternate embodiments, be more specifically defined by any of the transitional terms “comprising,” “consisting essentially of,” and “consisting of.”

While the presently disclosed subject matter has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the presently disclosed subject matter in its broader aspects is therefore not limited to the specific details, representative apparatus and method, and illustrative example shown and described. Accordingly, departures may be made from such details without departing from the spirit or scope of applicant's general inventive concept.

1. A device comprising:
a source configured to provide a beam of electromagnetic energy;
a collimator configured to receive the beam from the source;
a first optical element; and
a second optical element having an optical axis;
wherein the first optical element is positioned between the collimator and the second optical element, and the first optical element is configured to correct for misalignment of the beam relative to the optical axis of the second optical element.

2. The device of claim 1 wherein the first optical element comprises a first prism and a second prism positioned adjacent to the first prism.

3. The device of claim 2 wherein the first prism is a first cylinder having a first base, a second base opposite to the first base, and a cylindrical surface connecting the first base to the second base, and the first base of the first prism has a first wedge angle relative to the second base of the first prism.

4. The device of claim 3 wherein the second prism is a second cylinder having a first base, a second base opposite to the first base, and a cylindrical surface connecting the first base to the second base, and the first base of the second prism has a second wedge angle relative to the second base of the second prism.

5. The device of claim 4 wherein the first wedge angle is equal to the second wedge angle.

6. The device of claim 4 wherein the first wedge angle and the second wedge angle are greater than 0 degrees and less than or equal to about 3 degrees.

7. The device of claim 4 wherein the first wedge angle and the second wedge angle are equal to about 0.5 degrees.

8. The device of claim 3 wherein the second base of the first prism is arranged adjacent to the second base of the second prism.

9. The device of claim 3 wherein the beam of electromagnetic energy incident on the first base of the first prism.

10. The device of claim 2 further comprising:
a plurality of clamps having a clamped state and an unclamped state;
a first sleeve configured to support the first prism, the first sleeve positioned inside the clamps; and
a second sleeve configured to support the second prism, the second sleeve placed inside the clamps.

11. The device of claim 10 wherein the first sleeve and the second sleeve are rotatable when the clamps are in the unclamped state, and the first sleeve and the second sleeve are fixed when the clamps are in the clamped state.

12. The device of claim 10 wherein the first sleeve includes a plurality of slots that are engageable by a tool for rotating the first sleeve and the first prism relative to the clamps when the clamps are in the unclamped state.

13. The device of claim 12 wherein the first prism has an optical axis, and the first prism has an axis of rotation that coincides with the optical axis.

14. The device of claim 10 further comprising:
a plurality of fasteners connecting different portions of the clamps, the fasteners configured to be tightened to establish the clamped state and loosened to establish the unclamped state.

15. The device of claim 1 further comprising:
a handpiece configured and dimensioned to be manually grasped,
wherein the collimator, the first optical element, and the second optical element are positioned inside the handpiece.

16. The device of claim 1 wherein the source is a laser, and further comprising:
an optical fiber connecting the source with the collimator.

17. The device of claim 16 wherein the source comprises laser diode.

18. The device of claim 1 wherein the source is a fiber-coupled laser diode that is terminated by the collimator.

19. The device of claim 1 wherein further comprising:
a scanner wheel configured to spin about an axis of rotation, the scanner wheel having a plurality of reflective elements arranged about a circumference that are configured to receive the beam from the second optical element and generates a plurality of beamlets from the beam that are output from the scanner wheel in a pattern.

20. A method comprising:
directing a beam of electromagnetic energy though a collimator to a first optical element; and
rotating a first prism of the first optical element relative to a second prism of the first optical element to correct for misalignment of the beam relative to an optical axis of a second optical element downstream from the first optical element.
